(19) United States
(12) Patent Application Publication
(10) Pub. No.: US 2011/0189710 A1
(43) Pub. Date: Aug. 4, 2011

(54) TREATMENT OF POMPE DISEASE WITH SPECIFIC PHARMACOLOGICAL CHAPERONES AND MONITORING TREATMENT USING SURROGATE MARkERS

(75) Inventors: Brandon Wustman, San Diego, CA (US); Hung V. Do, New Hope, PA (US)

(73) Assignee: AMICUS THERAPEUTICS, INC., Cranbury, NJ (US)

(21) Appl. No.: 12/920,864

(22) PCT Filed: Mar. 12, 2009
(86) PCT No.: PCT/US09/36936

§ 371 Date: Mar. 29, 2011

Related U.S. Application Data
(60) Provisional application No. 61/035,869, filed on Mar. 12, 2008.

(51) Int. Cl. C12Q 1/34; C12Q 1/02

(52) U.S. Cl. 435/18; 435/29

(57) ABSTRACT

Provided is a method of monitoring the treatment of Pompe disease with specific pharmacological chaperones using systemic and/or cellular surrogate markers.

### CROSS-REFERENCE TO RELATED APPLICATIONS

This application claims priority to U.S. Provisional Application No. 61/035,869 filed Mar. 12, 2008; the contents of which are incorporated herein by reference.

### FIELD OF THE INVENTION

The present invention provides a method for monitoring the treatment of an individual having Pompe disease with a specific pharmacological chaperone by determining the presence and levels of specific surrogate markers such as α-glucosidase, cathepsins, growth factors and cytokines. The present invention also provides a method for monitoring the treatment of an individual having Pompe disease with a specific pharmacological chaperone by evaluating the effects of treatment at the cellular level.

### BACKGROUND

Pompe disease is an inherited metabolic disorder that is one of approximately forty lysosomal storage disorders (LSDs). These LSDs are a group of autosomal recessive diseases caused by the accumulation of cellular glycosphingolipids, glycogen, or mucopolysaccharides, due to defective hydrolytic enzymes. Examples of lysosomal disorders include but are not limited to Gaucher disease (Beutler et al., The Metabolic and Molecular Bases of Inherited Disease, 8th ed. 2001 Scriver et al., ed. pp. 3635-3668. McGraw-Hill, New York). G_{M1}-gangliosidosis (id. at pp 3775-3810), fucosidosis (The Metabolic and Molecular Bases of Inherited Disease 1995. Scriver, C. R. Beaudet, A. E., Sly, W. S, and Valle, D., ed pp. 2529-2561, McGraw-Hill, New York), mucopolysaccharidoses (id. at pp 3421-3452), Pompe disease (id. at pp. 3389-3420), Hurler-Scheie disease (Weismann et al. Science. 1970; 169, 72-74), Niemann-Pick A and B diseases, (The Metabolic and Molecular Bases of Inherited Disease 8th ed. 2001. Scriver et al. Ed. pp 3589-3610, McGraw-Hill, New York), and Fabry disease (Id. at pp. 3733-3774).

The specific pharmacological chaperone (“SPC”) strategy has been demonstrated for numerous enzymes involved in lysosomal storage disorders as in U.S. Pat. Nos. 6,274,597, 6,583,158, 6,589,964, 6,599,919, and 6,916,829 to Fan et al. which are incorporated herein by reference in their entirety. For example, a small molecule derivative of galactose, 1-deoxygalactonojirimycin (DGJ), a potent competitive inhibitor of the mutant Fabry enzyme α-galactosidase A (α-Gal A; GLA), effectively increased in vitro stability of the human mutant α-Gal A (R301Q) at neutral pH, and it enhanced the mutant enzyme activity in lymphoblasts established from Fabry patients with R301Q or Q279E mutations. Furthermore, oral administration of DGJ to transgenic mice overexpressing a mutant (R301Q) α-Gal A substantially elevated the enzyme activity in major organs (Fan et al. Nature Med. 1999; 5: 112-115). Similar rescue of glucocerebrosidase (acid β-glucosidase, GBA) from Gaucher patient cells has been described using another iminosugar, isofagomine (IFG), and its derivatives, described in U.S. Pat. No. 6,916,829, and using other compounds specific for glucocerebrosidase (described in pending U.S. patent application Ser. Nos. 10/988,428, and 10/988,427, both filed Nov. 12, 2004). U.S. Pat. No. 6,583,158, described above, discloses several small molecule compounds that would be expected to stabilize mutant GAAs and increase cellular levels of the enzyme for the treatment of Pompe disease, including 1-deoxynojirimycin (DNJ). α-homonojirimycin, and castanospermine.

However, as indicated above, successful candidates for SPC therapy must have a mutation which results in the production of an enzyme that has the potential to be stabilized and folded into a conformation that permits trafficking out of the ER. Mutations which severely truncate the enzyme, such as nonsense mutations, or mutations within the catalytic domain which prevent binding of the chaperone, will not likely be “rescuable” or “enhanceable” using SPC therapy. However, it is often difficult to predict responsiveness of specific mutations even if they are outside the catalytic site and requires empirical experimentation. Moreover, since WBCs only survive for a short period of time in culture (ex vivo), screening for SPC enhancement of GAA is difficult.

Despite the phenotypic inconsistency, Pompe patients exhibit several consistent surrogate markers of the disease that are used to evaluate clinical response to treatment. The present invention relates to a method of monitoring treatment of a Pompe patient following treatment with a specific pharmacological chaperone, by evaluating changes in at least one, and preferably multiple, surrogate markers of Pompe disease.

### SUMMARY OF THE INVENTION

The present invention provides a method for monitoring treatment of a Pompe disease patient with a specific pharmacological chaperone for α-glucosidase (Gaa), by evaluating changes in the presence and/or level of a surrogate marker that is associated with Pompe disease, where an improvement indicates that the individual is responding to the chaperone therapy.

In one embodiment, the surrogate marker is a systemic surrogate marker.

Systemic surrogate markers include at least one of the following: decreased lysosomal Gaa activity in cells and urine; the presence of lipid-laden macrophages (“Pompe macrophages”); increased levels of cathepsin B, increased levels of Macrophage inflammatory protein 1 alpha (MIP-1 alpha), increased levels of vascular endothelial growth factor (VEGF), increased levels of Interleukin-6 increased levels of Interleukin-8 (IL-8), increased levels of Interleukin-17 (IL-17), increased levels of collagen IV, decreased levels of cathepsin D, decreased levels of hepatocyte growth factor (HGF), decreased levels of platelet-derived growth factor AA (PDGF-AA), decreased levels of platelet-derived growth factor AA/BB (PDGF-AA/BB), decreased levels of Interleukin-7 (IL-7), and decreased levels of Interleukin-12 p40 subunit (IL-12p40).

Additional surrogate markers include progressive muscle myopathy throughout the body which affects various body tissues, particularly the heart, skeletal muscles, liver, and nervous system; severe lack of muscle tone; weakness: enlarged liver and heart; cardiomyopathy; difficulty in swallowing; protrusion and/or enlargement of the tongue; respiratory myopathy, weakness in the muscles of the diaphragm, trunk and/or lower limbs

In a specific embodiment, the combination of markers expected following treatment of Pompe disease with a pharmacological chaperone are as follows: increased α-glucosidase (Gaa) levels in white blood cells, skin and urine; decreased glycosphingolipids, glycogen, and/or mucopolysaccharides levels in white blood cells, plasma, serum, urine and skin; decreased levels of cathepsin B in plasma, decreased levels of Macrophage inflammatory protein 1 alpha (MIP-1 alpha) in plasma, decreased levels of vascular endothelial growth factor (VEGF) in plasma, decreased levels of interleukin-6 (IL-6) in plasma, decreased levels of Interleukin-8 (IL-8) in plasma, decreased levels of Interleukin-17 (IL-17) in plasma, decreased levels of collagen IV in plasma, increased levels of cathepsin D in plasma, increased levels of hepatocyte growth factor (HGF), increased levels of platelet-derived growth factor AA (PDGF-AA) in plasma, increased levels of platelet-derived growth factor AA/BB (PDGF-AA/BB) in plasma, increased levels of Interleukin-7 (IL-7) in plasma, and increased levels of Interleukin-12 p40 subunit (IL-12p40) in plasma.

In another embodiment, the surrogate marker is a sub-cellular surrogate marker.

Sub-cellular surrogate markers include at least one of the following: aberrant trafficking of Gaa in cells from Pompe patients from the ER to the lysosome; aberrant trafficking of cellular lipids though the endosomal pathway: the presence of increased amounts misfolded Gaa in the ER or cytosol: the presence of ER and/or stress resulting from toxic accumulation of Gaa (as determined by gene and/or protein expression of stress-related markers): aberrant endosomal pH levels: the presence of increased plasma membrane expression of MHCII and/or CD1d on monocytes: aberrant cell morphology: suppression of the ubiquitin/proteasome pathway: and an increase in the amount of ubiquitinated proteins.

In a further embodiment, the specific pharmacological chaperone used in the therapy is an inhibitor of Gaa, such as a reversible competitive inhibitor.

In specific embodiments, the inhibitor is 1-deoxynojirimycin (DNJ).

The present invention also provides a method for treating Pompe disease with effective amount of a specific chemical chaperone that binds to Gaa, and monitoring its effect on cytoplasmic staining of cells, where restoration of an abnormal staining pattern indicates that the individual with Pompe disease is responding to chaperone treatment. In one embodiment, the cytoplasmic staining is lysosomal staining, in particular, detection of Gaa or LAMP-1 expression in the lysosome.

In another embodiment, the cytoplasmic staining is detection of polyubiquitinated proteins.

In a particular embodiment, the specific pharmacological chaperone is an inhibitor of Gaa, such as a reversible competitive inhibitor.

In specific embodiment, the inhibitor is DNJ.

### BRIEF DESCRIPTION OF THE DRAWINGS

This patent application contains at least one drawing executed in color. Copies of this patent or patent application publication with color drawing(s) will be provided by the Office upon request and payment of the necessary fee.

FIG. 1. FIG. 1 depicts the effects of 1-DNJ, NB-DNJ and N-(cyclopropyl)methyl DNJ iminosugar derivatives on the activity of acid α-glucosidase in the Pompe disease cell line PM-11.

FIGS. 2A-D. FIG. 2 shows Gaa enhancement in brain (2A), liver (2B), gastrocnemius (2C), and tongue (2D) of normal C57BL6 mice treated with various concentrations of DNJ and NB-DNJ for 2 weeks.

FIGS. 3A-D. FIG. 3 shows Gaa enhancement in kidney (3A), diaphragm (3B), heart (3C), and soleus (3D) of normal C57BL6 mice treated with various concentrations of DNJ and NB-DNJ for 2 weeks.

FIGS. 4A-D. FIG. 4 shows Gaa enhancement in brain (4A), liver (4B), gastrocnemius (4C), and tongue (4D) of normal C57BL6 mice treated with various concentrations of DNJ and NB-DNJ for 4 weeks.

FIGS. 5A-D. FIG. 5 shows Gaa enhancement in kidney (5A), diaphragm (5B), heart (5C), and soleus (5D) of normal C57BL6 mice treated with various concentrations of DNJ and NB-DNJ for 4 weeks.

FIGS. 6A-H. FIG. 6 depicts Gaa immunostaining in wild-type (6C) and Pompe PM8 (6A and 6F) fibroblasts. This figure also depicts lysosomal staining for lysosomal marker LAMP-1 in wild-type (6D) and Pompe PM8 fibroblasts (6B and 6E). An overlay of Gaa and LAMP-1 staining for wild-type (6H) and PM8 (6G) fibroblasts is also shown.

FIGS. 7A-F. FIG. 7 depicts immunofluorescent staining for Gaa (7B and D) and LAMP-1 (7E) in PM9 Pompe fibroblasts. Overylays of Gaa and LAMP-1 staining are also depicted (7A, 7C and 7F).

FIG. 8. FIG. 8 depicts Gaa. LAMP-1, and Gaa/LAMP-1 dual staining PM11 Pompe cell lines that have been treated with DNJ or NB-DNJ.

FIG. 9. FIG. 9 depicts the concentration of cathepsin B, cathepsin B, PDGF-AA, PDGF-AA/BB, MIP-1 alpha. VEGF. IL-6, IL-7, IL-8. IL-12p40, IL-17 and collagen IV in plasma from Pompe patients as compared to plasma from controls.

### DETAILED DESCRIPTION

The present invention demonstrates a response to treatment with SPCs in a Pompe disease model as evidenced by evaluation of specific surrogate markers of Pompe disease following treatment. Accordingly, the present invention provides standards of care for evaluating response to SPC treatment in Pompe patients by evaluating the patient for changes, i.e., improvements, in specific surrogate markers.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Pompe disease” also referred to as acid maltase deficiency, glycogen storage disease type II (GSDII), and glycogenosis type II, is a genetic lysosomal storage disorder characterized by mutations in the Gaa gene which metabolizes glycogen. As used herein, this term includes infantile, juvenile and adult-onset types of the disease.

A “patient” refers to a subject who has been diagnosed with a particular disease. The patient may be human or animal. A “Pompe disease patient” refers to an individual who has been diagnosed with Pompe disease and has a mutated Gaa as defined further below.

As used herein the term “mutant α-glucosidase” or “mutant Gaa” refers to an α-glucosidase polypeptide translated from a gene containing a genetic mutation that results in an altered α-glucosidase amino acid sequence. In one embodiment, the mutation results in an α-glucosidase protein that does not achieve a native conformation under the conditions normally present in the ER, when compared with wild-type α-glucosidase or exhibits decreased stability or activity as compared with wild-type α-glucosidase. This type of mutation is referred to herein as a “conformational mutation,” and the protein bearing such a mutation is referred as a “conformational mutant.” The failure to achieve this conformation results in the α-glucosidase protein being degraded or aggregated, rather than being transported through a normal pathway in the protein transport system to its native location in the cell or into the extracellular environment. In some embodiments, a mutation may occur in a non-coding part of the gene encoding α-glucosidase that results in less efficient expression of the protein, e.g., a mutation that affects transcription efficiency, splicing efficiency, mRNA stability, and the like. By enhancing the level of expression of wild-type as well as conformational mutant variants of α-glucosidase, administration of an α-glucosidase pharmacological chaperone can ameliorate a deficit resulting from such inefficient protein expression. Alternatively, for splicing mutants or nonsense mutants which may accumulate in the ER, the ability of the chaperone to bind to and assist the mutants in exiting the ER, without restoring lysosomal hydrolase activity, may be sufficient to ameliorate some cellular pathologies in Pompe patients, thereby improving symptoms.

Exemplary conformational mutations of Gaa include the following: D645E (Lin et al., Zhonghua Min Gun Xiao Er Ke Yi Xue Hui Za Zhi. 1996; 37(2):115-21); D64511 (Lin et al. Biochem Biophys Res Commun. 1995 17; 208(2):886-93); R224W, S619R, and R660H (New et al. Pediatr Neurol. 2003; 29(4):284-7); T1064C and C2104T (Montalvo et al., Mol Genet Metab. 2004; 81(3):203-8); D645N and L901Q (Kroos et al., Neuromuseul Disord. 2004; 14(6):371-4): G219R, E262K, M408V (Fernandez-Hojas et al., Neuromuscul Disord. 2002:12(2):159-66); G309R (Kroos et al., Clin Genet. 1998; 53(5):379-82): D645N, G448S, R672W, and R672Q (Huie et al., Biochem Biophys Res Commun. 1998; 27; 244(3):921-7); P545L (Hermans et al. Hum Mol. Genet. 1994; 3(12):2213-8); C647W (Huie et al., Huie et al., Hum Mol Genet. 1994:3(7):1081-7): G643R (Hermans et al. Hum Mutat. 1993:2(4):268-73): M318T (thong et al., Am J Hum Genet. 1991; 49(3):635-45); F521K (Hermans et al., Biochem Biophys Res Commun. 1991; 179(2):919-26); W481R (Raben et al. Hum Mutat. 1999; 13(1):83-4); and L552P and G549R (unpublished data).

Splicing mutants include IVS1AS, T>G, −13 and IVS8+1G>A).

Additional Gaa mutants have been identified and are known in the art. Conformational mutants are readily identifiable by one of ordinary skill in the art.

Mutations which impair folding, and hence, trafficking of Gaa, can be determined by routine assays well known in the art, such as pulse-chase metabolic labeling with and without glycosidase treatment to determine whether the protein enters the Golgi apparatus, or fluorescent immunostaining for Gaa localization within the cell. Wild-type Gaa is secreted as a 110 kD precursor which then converts to the mature Gaa of 76 kD via and intermediate of 95 kD.

Such functionality can be tested by any means known to establish functionality of such a protein. For example, assays using fluorescent substrates such as 4-methyl umbeliferryl-α-D-ducopyranoside can be used to determine Gaa activity. Such assays are well known in the art (see e.g., Hermans et al. above).

As used herein, the term “specific pharmacological chaperone” (“SPC”) refers to any molecule including a small molecule, protein, peptide, nucleic acid, carbohydrate, etc. that specifically hinds to a protein and has one or more of the following effects: (i) enhancing the formation of a stable molecular conformation of the protein; (ii) inducing trafficking of the protein from the ER to another cellular location, preferably a native cellular location. i.e., preventing ER-associated degradation of the protein; (iii) preventing aggregation of misfolded proteins; and/or (iv) restoring or enhancing at least partial wild-type function and/or activity to the protein. A compound that specifically binds to e.g. Gaa, means that it binds to and exerts a chaperone effect on Gaa and not a generic group of related or unrelated enzymes. Following is a description of some specific pharmacological chaperones contemplated by this invention:

1-deoxynojirimycin (DNJ) refers to a compound having the following structures:

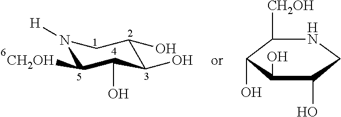

This term includes both the free base and any salt forms.

Still other SPCs for Gaa are described in U.S. Pat. No. 6,599,919 to Fan et al., and U.S. Patent Application Publication US 20060264467 to Mugrage et al. both of which are herein incorporated by reference in their entireties, and include N-methyl-DNJ, N-ethyl-DNJ, N-propyl-DNJ, N-butyl-DNJ, N-pentyl-DNJ, N-hexyl-DNJ, N-heptyl-DNJ, N-octyl-DNJ, N-nonyl-DNJ. N-methylcyclopropyl-DNJ, N-methylcyclopentyl-DNJ, N-2-hydroxyethyl-DNJ, and 5-N-carboxypentyl DNJ.

A “surrogate marker” or “surrogate clinical marker” of Pompe disease refers to the abnormal presence of, increased levels of, abnormal absence of, or decreased levels of a biomarker that is associated with Pompe disease and that is a reliable indicator of Pompe disease (but is not associated width a healthy individual) either alone or in combination with other abnormal markers or symptoms of Pompe disease.

As non-limiting examples, surrogate markers of Pompe disease, include decreased lysosomal Gaa activity; the presence of lipid-laden macrophages (“Pompe macrophages”); increased levels of cathepsin B, increased levels of Macrophage inflammatory protein 1 alpha (MIP-1 alpha), increased levels of vascular endothelial growth factor (VEGF), increased levels of Interleukin-6 (IL-6), increased levels of Interleukin-8 (IL-8), increased levels of Interleukin-17 (IL-17), increased levels of collagen IV, decreased levels of cathepsin D, decreased levels of hepatocyte growth factor (HGF), decreased levels of platelet-derived growth factor AA (PDGF-AA), decreased levels of platelet-derived growth factor AA/BB (PDGF-AA/BB), decreased levels of Interleukin-7 (IL-7), and decreased levels of Interleukin-12 p40 subunit (IL-12p40).

Additional surrogate markers include progressive muscle myopathy throughout the body which affects various body tissues, particularly the heart, skeletal muscles, liver, and nervous system; severe lack of muscle tone; weakness: enlarged liver and heart; cardiomyopathy; difficulty in swallowing; protrusion and/or enlargement of the tongue; respiratory myopathy, weakness in the muscles of the diaphragm, trunk and/or lower limbs

Other surrogate markers are present at the sub-cellular level (“sub-cellular surrogate markers”) and include: aberrant trafficking of Gaa in cells from Pompe patients from the ER to the lysosome; aberrant trafficking of cellular lipids though the endosomal pathway; the presence of increased amounts misfolded Gaa in the ER or cytosol; the presence of ER and/or stress resulting from toxic accumulation of Gaa (as determined by gene and/or protein expression of stress-related markers); aberrant endosomal pH levels; the presence of increased plasma membrane expression of MHCII and/or CD1d on monocytes; aberrant cell morphology: suppression of the ubiquitin/proteasome pathway; and an increase in the amount of ubiquitinated proteins.

An “an improvement in a surrogate marker” refers to an effect, following treatment with an SPC, of the amelioration or reduction of one or more clinical surrogate markers which are abnormally present or abnormally elevated in Pompe disease, or the presence or increase of one or more clinical surrogate markers which are abnormally decreased or absent in Pompe disease, relative to a healthy individual who does not have Pompe disease, and who does not have an other disease that accounts for the abnormal presence, absence, or altered levels of that surrogate marker.

A “responder” is an individual diagnosed with a disease associated with a Gaa mutation which causes misfolding of the Gaa protein, such as pompe disease, and treated according to the presently claimed method who exhibits an improvement in, amelioration of, or prevention of, one or more clinical symptoms, or improvement in one or more surrogate markers referenced above.

In addition, a determination whether an individual is a responder can be made at the sub-cellular level by evaluating improvements in the sub-cellular surrogate markers, e.g., intracellular trafficking of the mutant Gaa protein in response to treatment with an SPC. Restoration of trafficking from the ER is indicative of a response. Other sub-cellular evaluations that can be assessed to determine if an individual is a responder include improvements in the above-referenced sub-cellular surrogate markers.

The terms “therapeutically effective dose” and “effective amount” refer to the amount of the specific pharmacological chaperone that is sufficient to result in a therapeutic response. A therapeutic response may be any response that a user (e.g., a clinician) will recognize as an effective response to the therapy, including improvements in the foregoing symptoms and surrogate clinical markers. Thus, a therapeutic response will generally be an amelioration of one or more symptoms of a disease or disorder, such as those described above.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

### Formulations, Dosage, and Administration

DNJ and derivatives can be administered in a form suitable for any route of administration, including e.g., orally in the form tablets, capsules, or liquid, or in sterile aqueous solution for injection. In a specific embodiment, the DNJ (e.g. DNJ hydrochloride) is administered as a powder-filled capsule. When the compound is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants potato starch or sodium starch glycolate); or wetting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., water, sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled or sustained release of the ceramide-specific glucosyltransferase inhibitor.

The pharmaceutical formulations of DNJ or derivatives suitable for parenteral/injectable use generally include sterile aqueous solutions, or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating DNJ or derivatives in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The above formulations can contain an excipient or excipients. Pharmaceutically acceptable excipients which may be included in the formulation are buffers such as citrate buffer, phosphate buffer, acetate buffer, and bicarbonate buffer, amino acids, urea, alcohols, ascorbic acid, phospholipids, proteins, such as serum albumin, collagen, and gelatin; salts such as EDTA or EGTA, and sodium chloride; liposomes; polyvinylpyrollidone; sugars such as dextran, mannitol, sorbitol, and glycerol; propylene glycol and polyethylene glycol (e.g., PEG-4000, PEG-6000); glycerol, glycine or other amino acids and lipids. Buffer systems for use with the formulations include citrate, acetate, bicarbonate, and phosphate buffers. Phosphate buffer is a preferred embodiment.

The formulations can also contain a non-ionic detergent. Preferred non-ionic detergents include Polysorbate 20, Polysorbate 80, Triton X-100. Triton X-114. Nonidet P-40. Octyl α-glucoside, Octyl β-glucoside, Brij 35, Pluronic, and Tween 20.

### Administration

The route of administration of DNJ or derivatives may be oral (preferably) or parenteral, including intravenous, subcutaneous, intra-arterial, intraperitoneal, ophthalmic, intramuscular, buccal, rectal, vaginal, intraorbital, intracerebral, intradennal, intraeranial, intraspinal, intraventricular, intrathecal, intracisternal, intracapsular, intrapulmonary, intranasal, transmucosal, transdermal, or via inhalation.

Administration of the above-described parenteral formulations of DNJ or derivatives may be by periodic injections of a bolus of the preparation, or may be administered by intravenous or intraperitoneal administration from a reservoir which is external (e.g. an i.v. bag) or internal (e.g., a bioerodable implant). See, e.g., U.S. Pat. Nos. 4,407,957 and 5,798,113, each incorporated herein by reference. Intrapulmonary delivery methods and apparatus are described, for example, in U.S. Pat. Nos. 5,654,007, 5,780,014, and 5,814,607, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch. Needle-less injector devices are described in U.S. Pat. Nos. 5,879,327; 5,520,639; 5,846,233 and 5,704,911, the specifications of which are herein incorporated by reference. Any of the formulations described above can be administered using these methods.

Furthermore, a variety of devices designed for patient convenience, such as refillable injection pens and needle-less injection devices, may be used with the formulations of the present invention as discussed herein.

### Dosage

Persons skilled in the art will understand that an effective amount of the DNJ or derivatives used in the methods of the invention can be determined by routine experimentation. As a non-limiting example, the doses and regimens expected to be sufficient to increase Gaa in most “rescuable” individuals is as described in U.S. Provisional Application 61/028,105, filed Feb. 12, 2008, herein incorporated by reference in its entirety.

### Pompe Disease Treatment Monitoring Using Surrogate Markers

The present invention provides a method for monitoring the treatment of Pompe patients with specific pharmacological chaperones. Specifically, various assays are employed to evaluate the progress of the disease and its response to treatment with DNJ. In particular, various systemic and sub-cellular markers can be assayed. The monitoring aspect of the present invention encompasses both invasive and non-invasive measurement of various cellular substances.

Glycosphingolipids, glycogen, or mucopolysaccharides, Glycosphingolipids, glycogen, and mucopolysaccharides are compounds that pathologically accumulates in Pompe patients. Levels can be measured in urine and in plasma and tissues using a variety of accepted methods. In addition, one prevalent Pompe surrogate marker is the presence of the “Pompe macrophage.” The Pompe macrophage is an enlarged, lipid-laden macrophage that has a distinct morphology indicative of an activated macrophage.

Acid α-glucosidase activity. Decreased Gaa is associated with Pompe disease. As indicated above, non-invasive assessment of Gaa activity can be evaluated of peripherally obtained lymphoblasts, leukocytes and polymorphonuclear cells (PMNs) derived from Pompe patients. Cultured fibroblasts from skin biopsies can also be used. Such assays typically involve extraction of blood leukocytes from the patient, lysing the cells, and determining the activity upon addition of a substrate such as 4-methyl umbeliferryl-α-D-glucopyranoside (4MU-alphaGlc) (see e.g., Hermans et al. Human Mutation 2004; 23; 47-56).

Flow cytometry can also be used to evaluate Gaa activity in patient cells (Lorincz et al. Blood. 1997; 189:3412-20: and Chan et al., Anal Biochem. 2004; 334(21:227-33). This method employs a fluorogenic Gaa substrate which can be loaded into cells by pinocytosis. The cells are then evaluated using conventional fluorescein emission optics. Levels of fluorescence correlate with the amount of Gaa activity.

Cell morphology. Ultrastructural analysis of blood leukocytes and PMNs has been described (Laslo et al., Acta Paediatr. Hung. 1987; 28: 163-73). Briefly, electron microscopy can reveal the pathology in vacuole formations in patients with Pompe disease. This method can also be used to determine the presence of Pompe macrophages.

Hematologic manifestations. Hematologic manifestations of Pompe disease include increased plasma levels of cathepsin B, increased levels of Macrophage inflammatory protein 1 alpha (MIP-1 alpha), increased levels of vascular endothelial growth factor (VEGF), increased levels of Interleukin-6 (IL-6), increased levels of Interleukin-8 (IL-8), increased levels of Interleukin-17 (IL-17), increased levels of collagen IV, decreased levels of cathepsin D, decreased levels of hepatocyte growth factor (HGF), decreased levels of platelet-derived growth factor AA (PDGF-AA), decreased levels of platelet-derived growth factor AA/BB (PDGF-AA/BB), decreased levels of Interleukin-7 (IL-7), and decreased levels of Interleukin-12 p40 subunit (IL-12p40).

Myopathy biomarkers. Additional surrogate markers include progressive muscle myopathy throughout the body which affects various body tissues, particularly the heart, skeletal muscles, liver, and nervous system; severe lack of muscle tone; weakness; enlarged liver and heart; cardiomyopathy; difficulty in swallowing: protrusion and/or enlargement of the tongue; respiratory myopathy, weakness in the muscles of the diaphragm, trunk and/or lower limbs are all biomarkers which can indicate the manifestation of Pompe disease.

Organomegaly. Physical examination of patients afflicted with or suspected to have Pompe disease usually reveals the presence of an enlarged heart and/or liver when compared to normal individuals. Ultrasonography of the abdomen or MR imaging can determine extent of organomegaly in Pompe patients.

It is to be understood that these markers can be used to monitor treatment only if they are identified to be abnormal prior to treatment. In addition, it is preferable that the abnormal elevation of or decrease of the markers be correlated with the presence of the disease, and not attributed to other causes or concomitant diseases such as liver disease, avascular necrosis, pulmonary or cardiovascular diseases.

### Molecular Biology Monitoring Assays to Detect Sub-Cellular Markers

Monitoring of treatment of Pompe disease with specific pharmacological chaperones can be done at the sub-cellular level in addition to the systemic or macroscopic level, described above. For example, disturbances in endosomal-lysosomal membrane trafficking of lipids to the Golgi complex are characteristic of lysosomal storage disease (Sillence at al., J Lipid Res. 2002; 43(11):1837-45). Accordingly, one way of monitoring treatment of Pompe would be to contact cells from patients with labeled lipid (BODIPY-LacCer), or cabled glycogen, and monitor its trafficking in endosomal structures. Pathological accumulation in endosomal structures, for example, would be indicative that the patient is not responding well to treatment.

As one example, pH-sensitive fluorescent probes that are endocytosed by the cells can be used to measure pH ranges in the lysosomes and endosomes (i.e. fluorescein is red at pH 5, blue to green at 5.5 to 6.5). Lysosome morphology and pH will be compared in wild type and chaperone treated and untreated patient cells. This assay can be run in parallel with the plate reader assay to determine the pH-sensitivity. For example. BODIPY-LacCer is trafficked to the Golgi in normal cells, but accumulates in the lysosomes of cells with lipid storage disorders. BODIPY-LacCer fluoresces green or red depending on the concentration in the membrane, and the green/red color ratio in the lysosome can be used to measure changes in concentration.

Living healthy cells and patient cells, treated and untreated with compounds, will be incubated with BODIPY-LacCer and the red/green color ratio can be measured by the FACS and/or confocal microscope and the staining pattern (lysosome vs. Golgi) can be determined using a confocal microscope.

Trafficking occurs in cells along pH gradients (i.e. ER pH about 7. Golgi pH about 6.2-7.0, trans-Golgi network pH about 6.0, early and late endosomes pH about 6.5, lysosomes pH about 4.5) and luminal and endosomal pH is disrupted in cells with trafficking defects such as Pompe cells. Accordingly, an assay to determine pH sensitivity in wild type. SPC-treated and untreated patient cells, if correlated to positive effects of pH on trafficking, can be used to monitor restoration of trafficking in Pompe patients. If patient cells are more sensitive to changes in pH, than it would be possible to create a screening assay for SPCs that reduce the cells pH sensitivity, restores lysosome morphology or function, or more generally restores normal trafficking.

In addition, mitigation of the trafficking defect can be assessed at the molecular level by determining co-localization of the deficient enzyme (Gaa) with a lysosomal marker such as Lyso-Tracker®. Localization of Gaa in the lysosome is evidence that trafficking from the ER to the lysosome is restored by treatment with the specific pharmacological chaperone. In brief, normal and patient cells, treated and untreated with SPCs, are fixed and stained with primary antibodies to the enzyme and endosome/lysosome markers (e.g., Rab7. Rab9, LAMP-1, LAMP-2, dystrophin-associated protein PAD) and fluorescently tagged secondary antibodies. The FACS and/or confocal microscope is used to quantify the amount of fluorescence due to the concentration of enzyme and other endocytic pathway markers, and the confocal microscope can be used to determine changes in staining patterns.

in addition, traditional biochemical methods, such as pulse-chase metabolic labeling combined with Endoglycosidase H treatment. Endo H only cleaves proteins which have acquired ER glycosylation (high mannose N-linked), i.e. which are localized ER, but will not cleave proteins that have made it out of the ER to the Golgi and have acquired additional glycosylation in the Golgi. Accordingly, the greater the level of Endo H sensitive Gaa, the more accumulation of the protein in the ER. If the Gaa has made it into the Golgi, the glycosidase PNGase F can be used to confirm whether the protein has exited the Golgi since it cleaves all N-linked sugars.

ER Stress. The toxic accumulation of misfolded proteins in the ER of cells, such as the misfolded Gaa in Pompe patients, often results in ER stress. This leads to induction of the cell stress response which attempts to resolve the disruption in cell homeostasis. Accordingly, measuring markers of ER stress in patients following treatment with the specific pharmacological chaperone provides another way to monitor the effects of treatment. Such markers include genes and proteins associated with the Unfolded Protein Response, which include BiP, IRE1, PERK/ATF4, ATF6, XBP1 (X-box binding factor 1) and JNK (c-Jun N-terminal kinase). One method to assess ER stress is to compare expression levels between wild type and Pompe patient cells, and also between SPC-treated and untreated cells. ER stress inducers (e.g., tunicamycin for the inhibition of N-glycosylation and accumulation of unfolded proteins in the ER, lacatcystin or H₂O₂) and stress relievers (e.g., cyclohexamide to inhibit protein synthesis) can be used as controls.

Another method contemplated for monitoring the ER stress response is via gene chip analysis. For example, a gene chip with a variety of stress genes can be used to measure expression levels and type of ER stress response (early, late, apoptosis etc.). As one example, the HG-U95A array can be used. (Affymetrix. Inc.).

Lastly, since prolonged ER stress can result in apoptosis and cell death, depending on the level of unfolded proteins in the ER, and the resulting stress level, cells will be more or less sensitive to ER stress inducers such as tunicamycin or proteasome inhibitors. The more sensitive the cells are to the stress inducers, the higher the number of apoptotic or dead cells is observed. Apoptosis can be measured using fluorescent substrates analogs for caspase 3 (an early indicator of apoptosis). FACS, confocal microscopy, and/or using a fluorescence plate reader (96 well format for high through put assays) to determine the percentage of cells positive for apoptosis or cell death (FACS and/or confocal microscopy), or fluorescence intensity can be measured relative to protein concentration in a 96 well format with a fluorescence plate reader.

Another response to ER stress resulting from toxic protein accumulation in the ER is suppression of the ubiquitin/proteasome pathway. This leads to a general disruption of the endocytic pathway (Rocca et al., Molecular Biology of the Cell. 2001; 12:1293-1301). Misfolded protein accumulation is sometimes correlated with increased amounts of polyubiquitin (Lowe et al., Acuropathol Appl Neurobiol. 1990; 16:281-91).

Proteasome function and ubiquitination can be assessed using routine assays. For example, evaluation of 26S proteasome function in living animals by imaging has been achieved ubiquitin-luciferase reporter for bioluminescence imaging (Luker et al., Nature Medicine, 2003, 9, 969-973). Kits for proteasome isolation are commercially available from, for example. Calbiochem (Cat. No. 539176). Ubiquitination can be examined by morphological studies using immunohistochemistry or immunofluorescence. For example, healthy cells and patient cells, treated and untreated with SPCs, can be fixed and stained with primary antibodies to ubiquitinated proteins and fluorescence detection of secondary antibodies by FACS and/or confocal microscopy will be used to determine changes in ubiquitinated protein levels.

Another assay to detect ubiquitinated proteins is AlphaScreenn™ (Perkin-Elmer). In this model, the GST moiety of a GST-UbCH5a fusion protein is ubiquitinated using biotin-Ubiquitin (bio-Ub). Following ubiquitin activation by E1, in the presence of ATP, bio-Ub is transferred to UbCH5a. In this reaction. UbCH5a acts as the carrier to transfer the bio-Ub to its tagged GST moiety. The protein which becomes biotinylated and ubiquitinated is then captured by anti-GST Acceptor and streptavidin. Donor beads resulting in signal generation. No signal will be generated in the absence of ubiquitination.

Lastly, an ELISA sandwich assay can be used to capture ubiquitinated mutant Gaa. The primary antibody to the Gaa (e.g., rabbit) would be absorbed to the surface, enzyme would be captured during an incubation with cell lysate or serum, then an antibody (e.g., mouse or rat) to ubiquitinated protein, with secondary enzyme-linked detection, would be used to detect and quantify the amount of ubiquitinated enzyme. Alternatively, the assay could be used to quantify the total amount of multi-ubiquitinated proteins in cell extract or serum.

### Combination Therapy

The therapeutic monitoring of the present invention is also applicable following treatment of patients with a combination of DNJ and derivatives and ERT or gene therapy. Such combination therapy is described in commonly-owned. U.S. patent application publication number 2004/0180419 (Ser. No. 10/771,236), and in U.S. patent publication 2004/0219132 (Ser. No. 10/781,356). Both applications are herein incorporated by reference in their entirety.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

### Enhancement of Gaa with DNJ and DNJ Derivatives

Experiments described below indicate that DNJ and DNJ derivative N-butyl-DNJ, known inhibitors of enzymes responsible for glycolipid synthesis, also can bind to and enhance the activity of mutant Gaa without inhibiting glycolipid synthesis.

### Methods

Cell culture and seeding. The PM11 (P545L). PM8 and PM12 (both slicing defect), fibroblast cell lines was used for enhancement experiments. These cells are fibroblasts isolated from a Pompe patient. Cells were seeded at about 5000 cells per well in 180 μL media in sterile black clear-bottom 96 well Costar plates and incubated for about 3-6 hours at 37° C. with 5% CO₂. Media consisted of DMEM with 10% FBS and 1% penicillin/streptomycin.

Drug Treatment. All test compounds are dissolved in 1:1 DMSO:H2O to a stock concentration of 100 mM. Serial dilutions of the cells using another sterile black clear-bottom Costar plate were performed as follows:

1. 20 μL of 1:1 DMSO:H₂0 and 180 μL media were added to rows 3-11, and row 1, columns E-11 for a concentration of 5% DMSO, 5% H₂0 in media.

2. 20 μL of 100 mM DNJ and 180 μL media were added to row 1, columns A-D for a concentration of 10 mM DNJ

3. 30 μL of each 100 mM stock solution to be tested were added to an appropriate well in row 2 along with 270 μL media for a concentration of 10 mM)

4. Row 1 was mixed up and down three times using multi-channel pipet.

5. Row 2 was mixed as above and 100 μL was transferred from row 2 to row 3. Row 3 was mixed as described above, and 100 μL was transferred to sequentially to each of rows 4 through 11 (row 12 is left blank) in order to generate serial three-fold dilutions.

4. 20 μL was transferred from serial dilution plate according to Table 1.

5. The plate was incubated at 37° C. 5% CO₂ for 6 days with day 1 equal to the day of dosing.

Enzyme activity assay. Cells were washed two times with 200 μL dPBS followed by the addition of 70 μL of substrate (2.11 mM 3 mM 4-MU-α-D-glu) in citrate-phosphate buffer (30 mM sodium citrate, 40 mM sodium phosphate dibasic, pH 4.0), and 2.5% DMSO to rows 1-12. Following incubation at 37° C. with 5% CO₂ for about 3 h, 70 μL of stop buffer (0.4 M glycine pH 10.8) was added to rows 1-12. The plate was read in a Victor² multilabel counter-Wallac fluorescent plate reader and the fluorescence at F460 nm was determined h at an excitation of 355 nm and emission of 460 nm using 1 second read time per well. Enzyme activity per μg of protein in the supernatant was calculated from the amount of fluorescence emitted, which is directly proportional to the amount of substrate hydrolyzed, and hence, the amount of Gaa activity in the lysate. The enhancement ratio is the Gaa activity in the presence of the DNJ derivative divided by the Gaa activity without the compound.

### Results

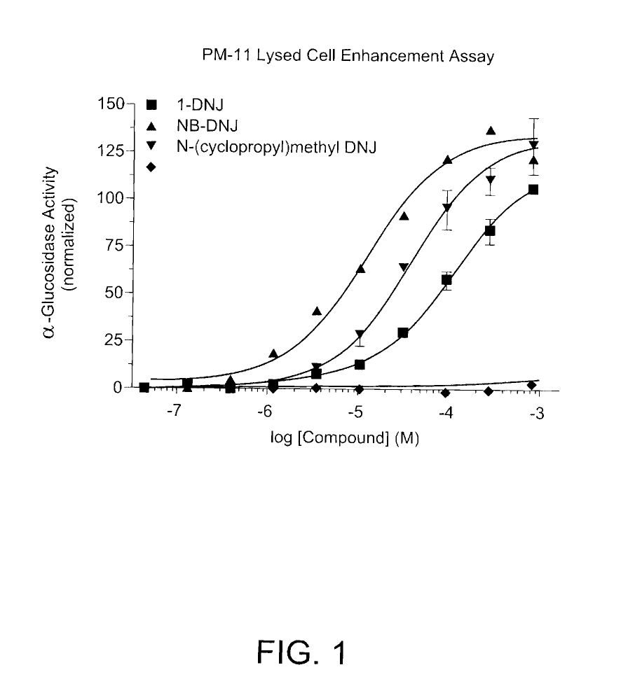

DNJ, NB-DNJ, and N-(cyclopropyl)methyl DNJ. As shown in FIG. 1, cells treated with DNJ (1), N-butyl-DNJ, (5) and N-(cyclopropyl)methyl DNJ (11), exhibited dose-dependent increases in Gaa activity compared to untreated control cells in the PM11 cell line. The highest concentration of DNJ, 1 mM, increases Gaa activity about 7.8-fold compared to Gaa activity in untreated cells (data not shown).

DNJ and NB-DNJ also significantly increased Gaa activity (more than 2-fold) in the PM12 cell lines at a concentration of 50 μM. No increases in Gaa activity in the PM8 cell line by DNJ were also observed (data not shown). Enhancement of Gaa by DNJ and NB-DNJ is dose-dependent, with increasing enhancement demonstrated at a range from 3.0-100 μM prior to plateau (data not shown).

Other DNJ Derivatives. As reported in Tables 1 and 2, below. DNJ derivatives N-methyl-DNJ, N-(2-(N,N-dimethylamido)ethyloxy-DNJ (15). N-4-t-butyloxycarbonyl-piperidnylmethyl-DNJ (16), N-2-R-tetrahydrofuranylmethyl-DNJ (17), N-2-R-tetrahydrofuranylmethyl-DNJ (18), N-(2-(2,2,2-trifluoroethoxy)ethyl-DNJ (19), N-2-methoxyethyl-DNJ (20), N-2-ethoxyethyl-DNJ (21), N-4-trifluoromethylbenzyl-DNJ (23), N-alpha-cyano-4-trifluoromethylbenzyl-DNJ (24), N-4-trifluoromethoxybenzyl-DNJ (25), N-4-n-pentoxybenzyl-DNJ (26), and N-4-n-butoxybenzyl-DNJ (27) also significantly increased Gaa activity in the PM-11. Increased Gaa activity using N-methyl DNJ and N-carboxypentyl DNJ was dose dependent from about 3-100 μM (data not shown).

% Eₘₐₓ refers to the percent maximal enhancement of an experimental compound relative to enhancement observed in the presence of 1 mM DNJ. It is calculated as the top of the theoretical nonlinear regression curve analyzed using GraphPad Prism version 3.02. Enhancement is defined as the average of multiple fluorescence counts normalized to the average maximum counts in the presence of 1 mM DNJ and to the minimum average counts in the absence of compound. Fluorescence counts were background subtracted. Background is defined by the average counts in the presence minus the absence of cells EC₅₀ (μM) refers to the concentration of compound that achieves 50% of Eₘₐₓ.

Without being limited to a particular mechanism, it is presumed that DNJ and the DNJ derivatives bind to mutant Gaa in the ER and induce a proper folding of the mutated protein, permitting the enzyme to exit the ER and traffic to the lysosome where it may exhibit some amount of enzymatic activity.

TABLE 1
N-ALKYL DERIVATIVES OF 1-DEOXYNOJIRIMYCIN

| Cmpd | | | EC₅₀ | % Eₘₐₓ |
|---|---|---|---|---|
| No. | Structure | Name | (μM) | (μM) |
| 1 | 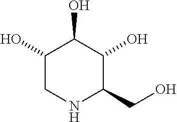 | DNJ | 98.8 ± 12.9 (n = 6) | 110.8 ± 3.5 (n = 6) |
| 2 | 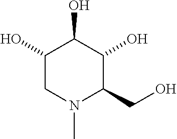 | N-Methyl-DNJ | 74.5 ± 9.5 (n = 3) | 67.3 ± 6.0 (n = 3) |
| 5 | 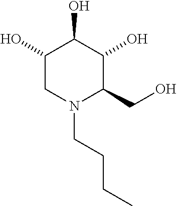 | N-Butyl-DNJ | 11.8 ± 2.2 (n = 6) | 138.9 ± 3.9 (n = 6) |
| 11 | 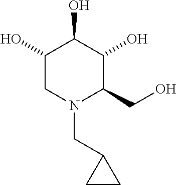 | N- (cyclopropyl)methyl DNJ | 47.7 ± 6.5 (n = 8) | 156.3 ± 4.5 (n = 8) |
| 15 | 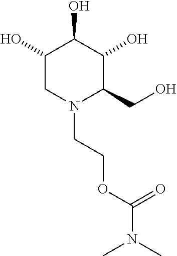 | N-ethyloxy DNJ dimethyl carbamate/N-(2- dimethylamido)ethyloxy) DNJ | 584.1 ± 89.9 (n = 3) | 50.6 ± 3.3 (n = 3) |
| 16 | 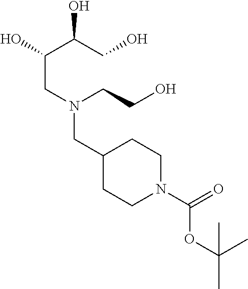 | 4-t-BOC- Piperidinylmethyl DNJ | 69.7 ± 9.7 (n = 3) | 80.0 ± 1.9 (n = 3) |
| 17 | 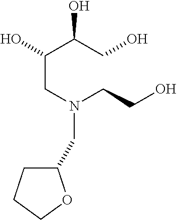 | N-2- (tetrahydrofuran) methyl DNJ | 653.2 ± 93.2 (n = 3) | 100.5 ± 3.0 (n = 3) |
| 18 | 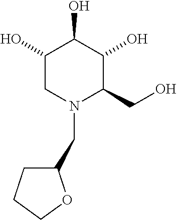 | N-2- (tetrahydrofuran) methyl DNJ | 103.5 ± 10.9 (n = 5) | 125.1 ± 6.9 (n = 5) |
| 19 | 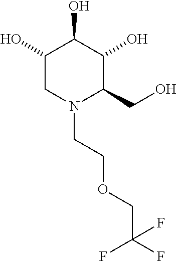 | N-2-oxoethyl DNJ trifluoro ether/ N-(2-(2,2,2- trifluoroethoxy) ethyl DNJ | 371.8 ± 43.1 (n = 3) | 170.2 ± 12.3 (n = 3) |
| 20 | 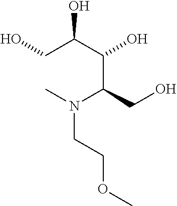 | 2-methoxyethyl DNJ | 467.7 ± 6.0 (n = 3) | 119.9 ± 10.5 (n = 3) |
| 21 | 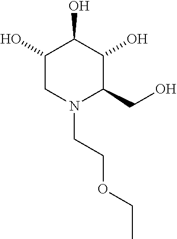 | 2-ethoxyethyl DNJ | 209.5 ± 13.1 (n = 3) | 115.0 ± −5.7 (n = 3) |
| 23 | 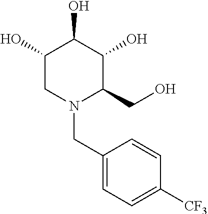 | 4-Trifluoromeethyl- benzyl DNJ | 121.0 ± 11.4 (n = 5) | 91.6 ± 7.5 (n = 5) |
| 24 | 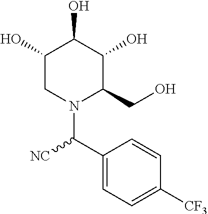 | α-cyano-4- Trifluoromethyl- benzyl DNJ | 77.1 ± 10.4 (n = 3) | 104.0 ± 6.8 (n = 3) |
| 25 | 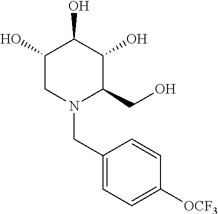 | 4- Trifluoromethoxy- benzyl DNJ | 66.5 ± 6.2 (n = 3) | 100.2 ± 6.3 (n = 3) |
| 26 | 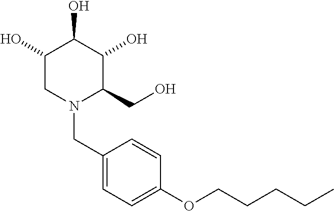 | 4-pentoxybenzyl DNJ | 6.6 ± 0.9 (n = 3) | 47.7 ± 3.9 (n = 3) |
| 27 | 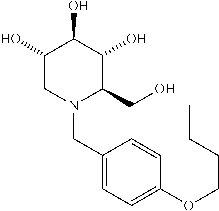 | 4-butoxybenzyl DNJ | 17.3 ± 1.6 (n = 3) | 68.5 ± 6.9 (n = 3) |

TABLE 2
DERIVATIVES OF 1-DEOXYNOJIRIMYCIN WITH C-
SUBSTITUTION

| Cmpd | | | EC₅₀ | % Eₘₐₓ |
|---|---|---|---|---|
| No. | Structure | Name | (μM) | (μM) |
| 32 | 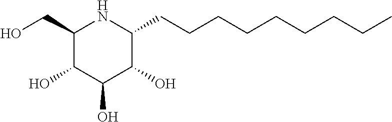 | α-C6-n-Nonyl- DNJ | 7.0 ± 1.8 (n = 5) | 38.9 ± 3.6 (n = 5) |
| 34 | 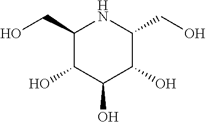 | α-homo-DNJ | 281.0 ± 95.2 (n = 3) | 58.2 ± 2.1 (n = 3) |

### Example 2

### In Vivo Gaa Activity Upon Treatment with DNJ and DNJ Derivatives

Drug administration. This Example provides information on the effects of DNJ derivatives on mice. The DNJ derivative test compounds were administered to the mice at 0, 1 mg/kg/day; 10 mg/kg/day; and 100 mg/kg/day; organs and plasma were collected at 2 and 4 weeks after initiation of the study. Twenty male C57BL6 (25 g) mice per group were used. The drug was given in the drinking water, therefore water consumption was monitored daily.

In the control group (0 mg/kg/day), the mice were dosed daily in the drinking water (no drug) and divided into two groups. Ten animals were euthanized after 2 weeks of treatment, blood was collected from the descending aorta or vena cava, and tissues were harvested and then necroposied. After 4 weeks of treatment, the remaining 10 animals were euthanized, and subjected to the same evaluation.

In the first test group, 20 mice were dosed daily in the drinking water with an administration aim of 1 mg/kg-day (assuming a 25 g mouse has daily drinking rate of 5 mL/day then the drinking water should have a concentration of 0.025 mg/5 ml or 5 micrograms/ml). Similar to the control, 10 mice were euthanized after 2 weeks of treatment and evaluated. After 4 weeks of treatment, the remaining 10 animals will be euthanized and evaluated.

For test compounds aiming for 10 mg/kg-day, 20 mice were dosed daily in the drinking water (estimating a compound concentration of 50 micrograms/ml) and divided into two groups for testing as described for the groups above.

For test compound at aiming for 100 mg/kg-day, 20 mice were dosed daily in the drinking water (estimating a compound concentration of 500 micrograms/ml) and divided into two groups were tested as described for the groups above.

The blood samples were drawn into lithium heparin and spun for plasma. After bleeding, the heart, liver, gastrocnemius muscle, soleus muscle, tongue, kidney, and brain were removed and placed into vials. The vials were put into dry ice for rapid freezing. The tissues and plasma were then analyzed for tissue levels of Gaa and glycogen.

Tissue preparation. Small portions of tissue were removed and added to 500 μl lysis buffer (20 mM sodium citrate and 40 mM disodium hydrogen phosphate, pH 4.0, including 0.1% Triton X-100). Tissues were then homogenized using a microhomogenizer for a brief time, followed by centrifugation at 10,000 rpt for 10 minutes at 4° C. Supernatants were transferred to a new tube and used for the enzyme assay.

Tissue enzyme assay. To 2.5 μl of supernatant (in 96-well plates) was added 17.5 μl reaction buffer (citrate phosphate buffer, no Triton), and 50 μl of 4-methyl umbel liferone (4-MU)-labeled substrate, α-glucopyranoside, or labeled negative controls. β-glueopyranoside and α-galacatopyranoside. Plates were incubated at 37° for 1 hour, followed by the addition of 70 μl stop buffer (0.4 M glycine-NaOH, pH 10.6). Activity of Gaa was determined by measuring the absorbance at 460 nm by exciting at 355 nm using a 1 second read time per well (Victor2 multilabel counter-Wallac) Enzyme activity was normalized to the amount in μl of lysate added, and enzyme activity per μl of lysate was estimated. The enhancement ratio is equal to the activity with the compound over the activity without the compound.

### Results

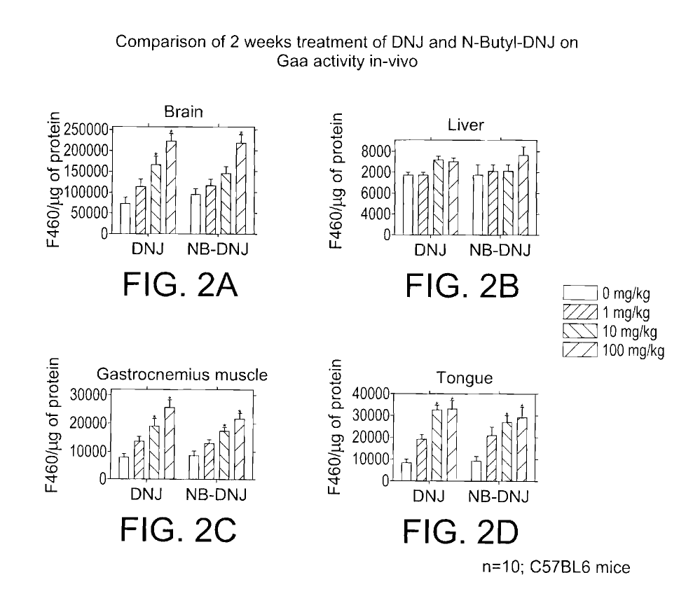

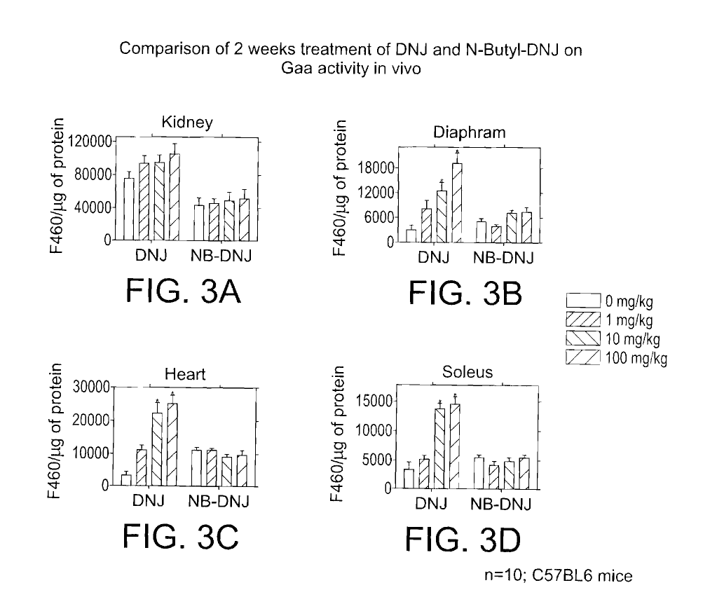

As demonstrated by FIGS. 2A-D and 3A-D. Gaa levels were increased following two weeks of treatment with DNJ and N-butyl-DNJ in the brain, liver, gastrocnemius muscle, tongue (FIGS. 2A-D), and also in the kidney, diaphragm, heart and soleus muscle (FIGS. 3A-D). The results were significant for a linear trend. For DNJ, the increases were dose-dependent in the brain, gastrocnemius muscle, tongue, kidney, diaphragm, heart, and soleus (significant for linear trend). For N-butyl-DNJ, the increases were dose-dependent in the brain liver, gastrocnemius muscle, tongue and kidney.

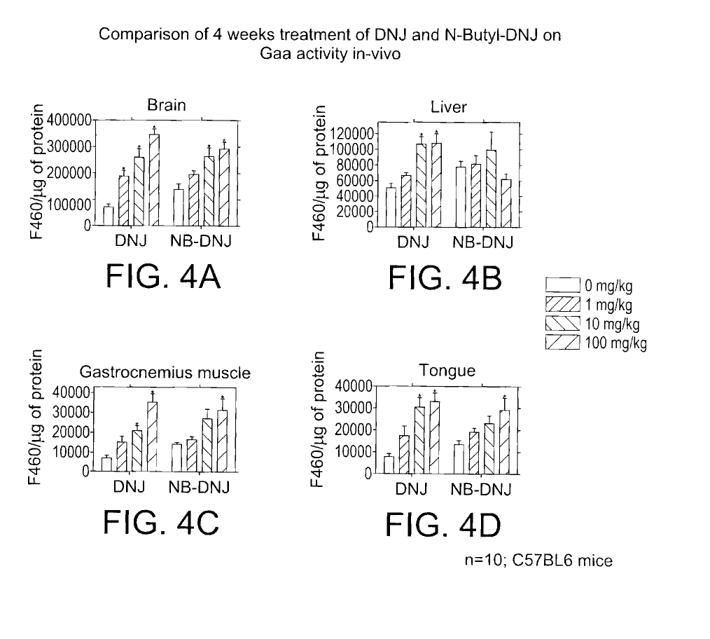

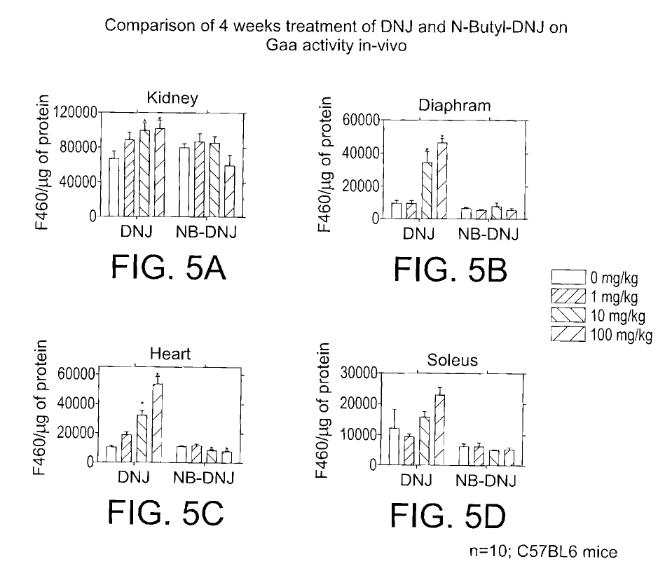

After 4 weeks of treatment. Gaa activity increases were observed following treatment with DNJ in the brain, liver, gastrocnemius muscle and tongue (FIGS. 4A-D), and also in the kidney, diaphragm, heart and soleus (FIGS. 5A-D). Results for N-butyl DNJ were similar except for the diaphragm, heart and soleus, where increases were not observed. Increases appeared to be dose-dependent in the brain, gastrocnemius muscle, tongue, kidney (DNJ only), diaphragm (DNJ only), heart (DNJ only) and soleus (DNJ only).

These results confirm that the specific pharmacological chaperones can increase the activity of non-mutated Gaa in vivo.

### Example 3

### Accumulation and Localization of Gaa with and without Exposure to DNJ Derivatives

In this experiment, four cell lines derived from Pompe patients who exhibited little to no residual Gaa activity were compared with wild-type fibroblasts for accumulation and localization of Gaa.

### Methods

Cell lines. PM8, PM9, PM11, and PM12 cell lines were evaluated. PM8 harbors a splicing defect resulting in some residual Gaa activity (IVS1AS, T>G, −13); PM9 harbors a nonsense mutation on one allele (R854X) and 3 missense mutations on the other (D645E, V816I, and T927I) and has essentially no residual Gaa activity (<1%): PM11 contains a missense mutation (P545L) and has some residual Gaa activity. PM12 also has a splicing defect (IVS8+G>A/M519V).

Immunofluorescence and microscopy. Cells cultured for 5 days with or without were grown for 5 days on glass coverslips with NB-DNJ. Cells were fixed with 3.7% paraformaldehyde for 15 minutes, permabilized with 0.5% saponin for 5 minutes, then labeled with a 1:300 dilution of rabbit anti-human Gaa (gift from Barry Byrne) and/or mouse monoclonal anti-LAMP1 (BD Pharmingen, catalog #555798) for 1 hour at room temperature. Secondary antibodies, goat anti-rabbit IgG conjugated with AlexaFluor 488, and goat-anti-mouse IgG conjugated with AlexaFluor 594 (Molecular Probes) were then added at a 1:500 dilution and incubated for 1 hour at room temperature. Coverslips were placed on slides with 10 μl Veetashield, sealed with fast-drying nail polish, and viewed with an 90i Nikon C1 confocal microscope.

### Results

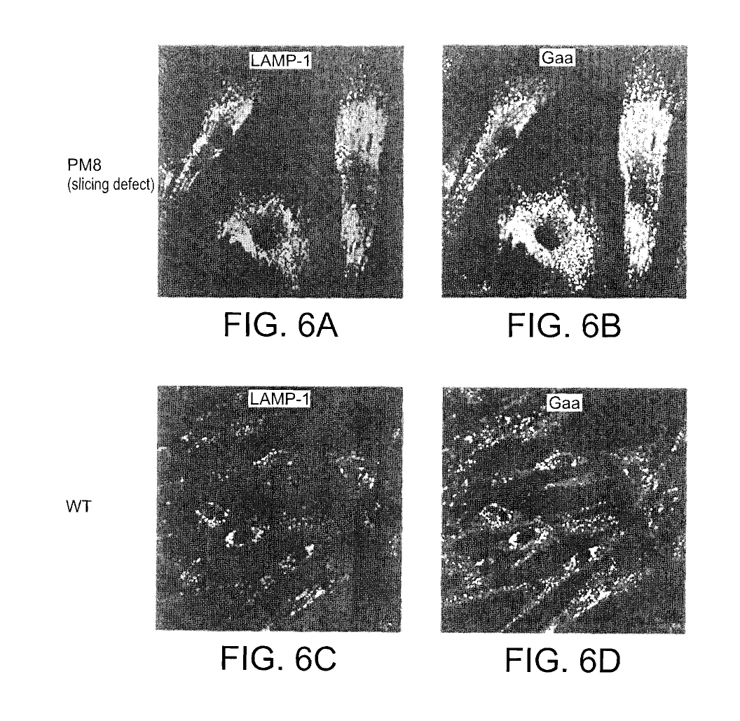

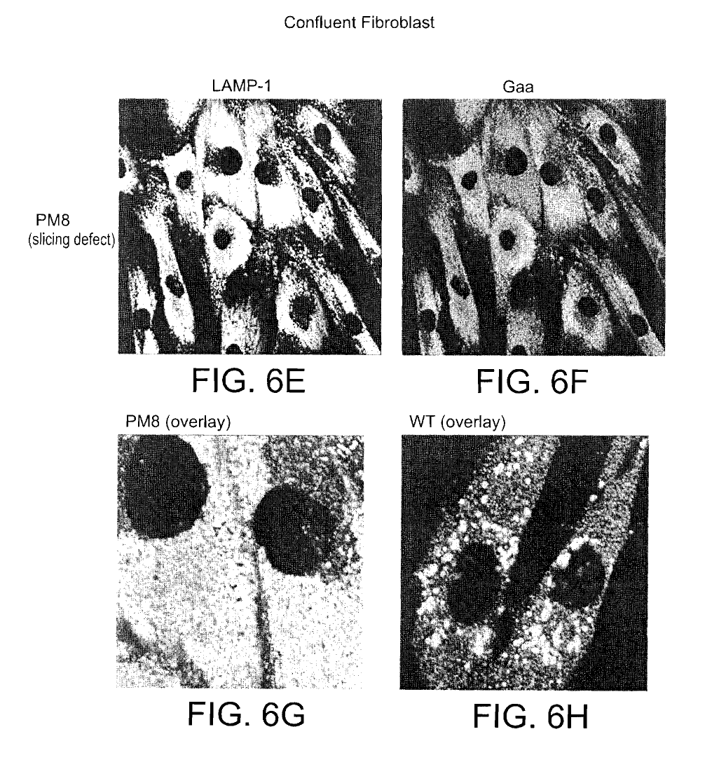

PM8. Despite having little residual Gaa activity. PM8 cells exhibited increased LAMP-1 and Gaa cytosolic staining, and had a different staining pattern, compared to wild-type fibroblasts. As shown in FIG. 6, wild-type fibroblasts treated with NB-DNJ exhibited a punctuate staining pattern for both LAMP-1 and Gaa (FIG. 6C-D), which appeared to co-localize in the lysosomes. By contrast, in the PM8 fibroblasts, staining was pervasive in the cytoplasm for both LAMP-1 and Gaa (FIGS. 6A-B and 6E-F). The overlay of both LAMP-1 and Gaa in confluent wild-type fibroblasts confirms co-localization to the lysosomes (FIG. 6H), whereas the overlay in confluent PM8 fibroblasts confirms the cytosolic excess of LAMP-1 and Gaa (FIG. 6G). The above results suggests a possible defect in lysosome formation or the presence of large aggregates of abnormally formed endosome/lysosome structures (aggresomes).

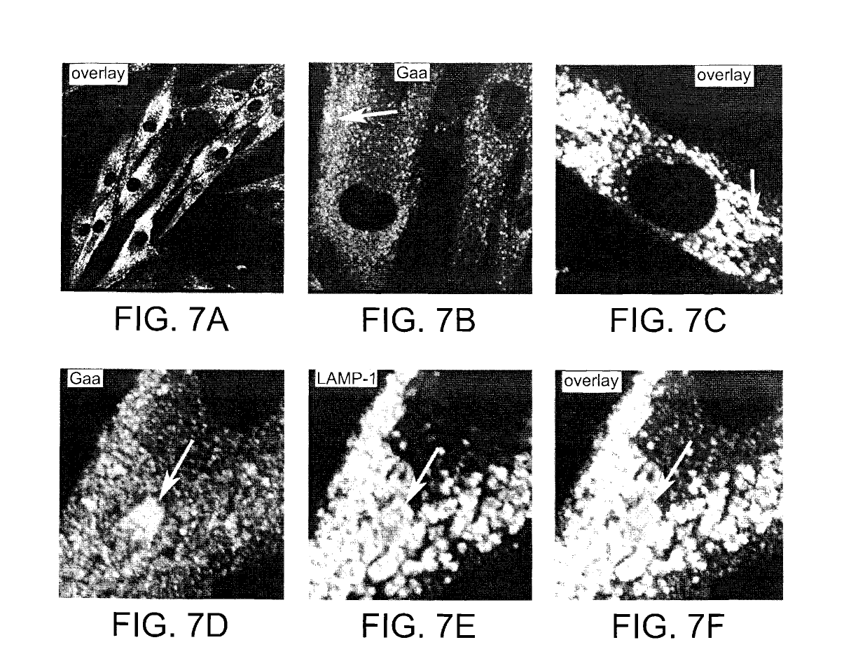

PM9. PM9 fibroblasts also exhibited an excess of Gaa (FIGS. 7B and 7D) and LAMP-1 (FIG. 7E) staining in the cytosol (FIG. 7B). An overlay shows the formation of Gaa aggregates that resemble aggresomes (FIGS. 7A, 7C and 7F, arrows and inlay show aggresomes). It is anticipated that treatment with DNJ derivatives will restore localization of Gaa to the lysosomes, and reduce aggresome formation. It is anticipated that treatment with DNJ derivatives will restore proper localization of Gaa to the lysosomes, and reduce the presence of cytosolic aggresomes.

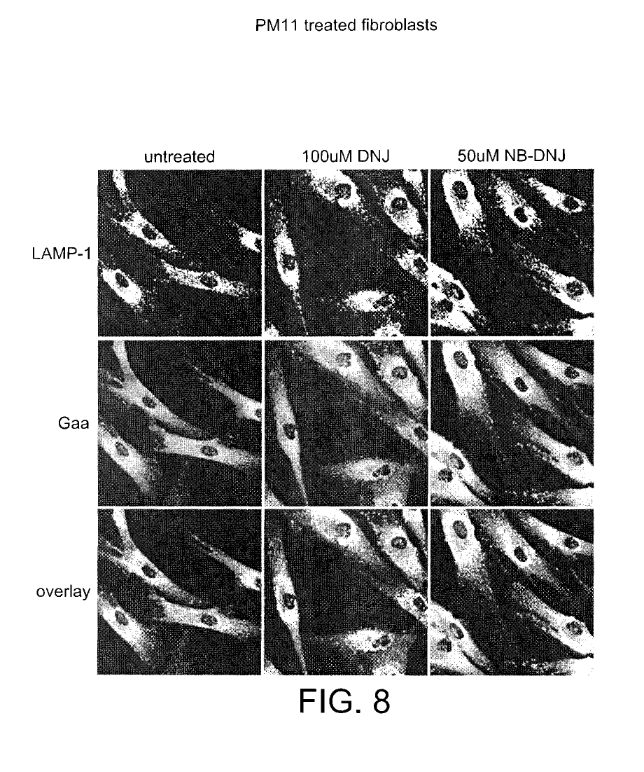

PM11. PM11 fibroblasts exhibit reduced Gaa activity. When treated with NB-DNJ (50 μM) and DNJ (100 μM), the PM11 cells exhibit an increase in intensity for labeling of Gaa in lysosomes as assessed by co-labeling with lysosmal marker LAMP-1, indicating restoration of trafficking (FIG. 8). Untreated PM11 fibroblasts exhibit some Gaa staining, little of which co-localizes with LAMP-1.

In addition, to confirm that the defect in PM11 cells is trafficking of lysosmal enzymes (Gaa) to the lysosomes, wild-type fibroblasts and PM11 cells were stained for early and late endosome markers EEA1 and M6PR, respectively. There was no difference in the localization patterns for early and late endosomes between wild type fibroblasts and Pompe PM11 fibroblasts (data not shown).

PM12. Significant increases in Gaa staining intensity was also observed in PM12 fibroblasts treated with NB-DNJ (data not shown).

### Discussion

This example demonstrates that the pharmacological chaperones of the present invention can restore the phenotype of cells harboring mutations in Gaa other than (and in addition to) those mutations which cause Gaa to become unstable and fail to exit the ER during synthesis. This supports a hypothesis where improving the trafficking of mutant Gaa from the ER to the lysosome may be sufficient to ameliorate some pathogenic effects of Pompe disease in tissues such as muscle, even without restoring Gaa hydrolase activity in the lysosome. It is clear that glycogen turnover is not enough to improve the patient phenotype in Pompe disease. Thus, one hypothesis for why improvements in trafficking may improve Pompe pathology is that lack of Gaa activity causes a glucose deficiency in cells, which may trigger or perpetuate an autophagic response (to use cytoplasmic glycogen for quick release of glucose). This autophagic response impairs trafficking through the endosomal trafficking pathways, resulting in the mistrafficking of membrane stabilizing proteins, and the ultimate breakdown of muscle fibers.

Chaperone therapy may rescue Gaa activity, alleviate the glucose deficiency and autophagic response induced by the glucose deficiency, and ultimately restore trafficking of membrane stabilizing proteins to prevent further muscle damage.

### Example 4

### Effect of DNJ Derivatives on Intestinal Gaa: Counterscreening

The ideal specific pharmacological chaperone, at sub-inhibitory concentrations, will enhance lysosomal Gaa without inhibiting intestinal Gaa. Accordingly, intestinal Gaa activity was evaluated in crude extracts from the mouse intestine at a pH of 7.0. In addition, an intestinal Gaa enzyme inhibition assay was established to determine whether compounds such as DNJ and NB-DNJ exerted an inhibitory effect on intestinal Gaa.

### Methods

Tissue preparation. Crude extracts were prepared from mouse intestines from C57BK6 mice as described above. Supernatants were transferred to a new tube and used for the enzyme assay.

### Results

DNJ was a more potent inhibitor of intestinal Gaa with an IC₅₀ value of 1 μM, while NB-DNJ had an IC₅₀ inhibitory value of 21 μM (data not shown).

### Example 5

### Treatment of Pompe Patients with DNJ Derivatives

In view of the results above, treatment of Pompe patients with the DNJ and DNJ derivatives of the present invention will reduce the pathologic accumulation of glycogen in muscle tissue, thereby ameliorating the disease state. In view of the fact that the currently approved sole treatment for Pompe disease, ERT, is ineffective in reducing glycogen accumulation in skeletal muscle since the recombinant enzyme cannot penetrate muscle tissue, this method solves a long-felt need in the art.

### Methods

Patient population. Patients with diagnosed infantile, juvenile and/or adult-onset Pompe disease will be recruited and evaluated in a randomized, double-blind, multiple-dose, open-label trial of orally administered DNJ derivative. In order to qualify, patients must have at least of the following: a) cardiomyopathy, defined as a left ventricular mass index (LVMI) determined by cross-sectional echocardiography; b) a requirement for invasive or non-invasive ventilatory support, where non-invasive ventilation is defined as any form of ventilatory support applied without the use of an endotracheal tube; or c) severe motor delay, defined as failure to perform gross motor skills achieved by 90% of normal aged peers on the Denver Developmental Screening Test (DDST-2: Hallioglo et al., Pediatr Int. 2001; 43(4):400-4).

Drug administration. Two groups of 10 subjects will receive either 50 or 100 mg of DNJ or a DNJ derivative twice a day for 24 weeks. This is below the amount indicated for substrate deprivation of glycosphingolipids in Gaucher disease.

Endpoints. Clinical efficacy will be evaluated by ventilator-free survival, left ventricular mass index, motor development and skeletal muscle function e.g., as measured using the Denver Developmental Screening Test and the Alberta Infant Motor Scale (Piper et al. Motor Assessment of the Developing Infant. Philadelphia. Pa., W.B. Saunders Co., 1994), the Bayley Scales of Infant Development II (BSIDII; Bayley et al., Bayley Scores of Infant Development. 2_{nd} Ed. San Antonio, Tex.: Harcourt Brace & Co. 1993), as well as histologic and biochemical analysis of muscle biopsies. i.e., a determination of glycogen levels in treated versus untreated patients using periodic acid-Schiff (PAS)-positive staining and enzyme activity assays, and measurement of Gaa activity in fibroblasts obtained from the patients. Clinical measurements will be assessed bi-weekly, except for muscle biopsies which will be assessed at 4, 12 and 24 weeks.

### Results

Treatment with a DNJ derivative will be effective for the treatment of Pompe disease by ameliorating some of the symptoms and reducing the muscle tissue levels of glycogen. For example, it is expected that within 12 weeks, increases in Gaa activity in muscle will be observed, and that the accumulation of glycogen in muscle will be reduced. In addition, it is expected that LVMI will be reduced and respiratory symptoms will improve. Lastly, progress in motor development and muscle tone, especially in young patients, is expected.

### Example 6

### Analysis of Surrogate Markers in Human Pompe Patients and Human Controls

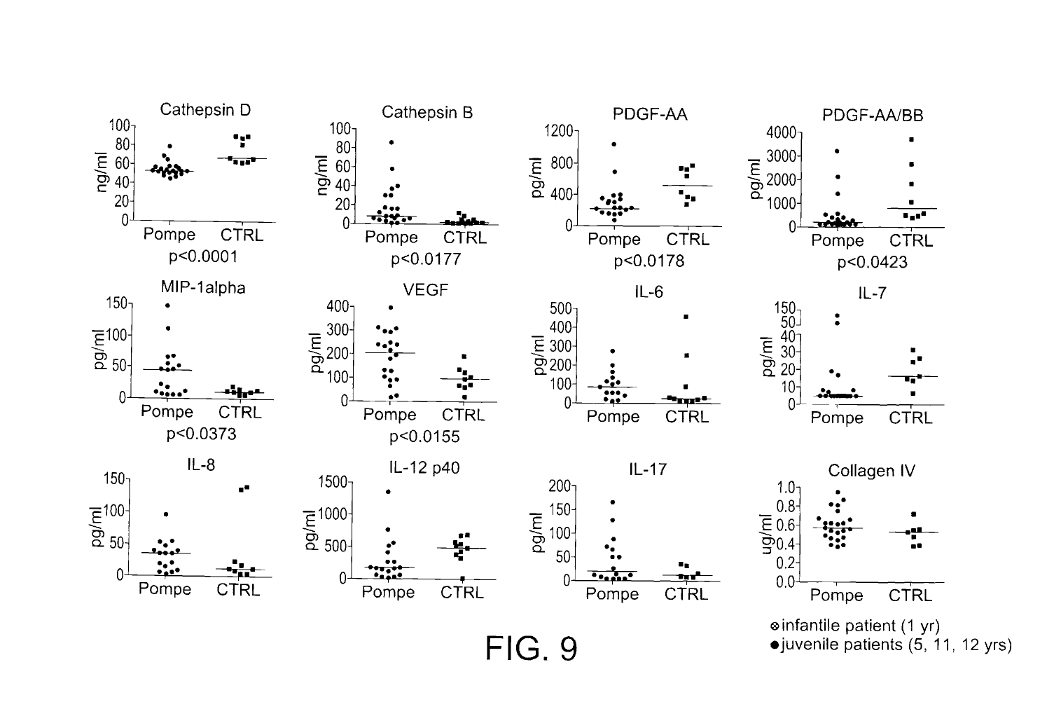

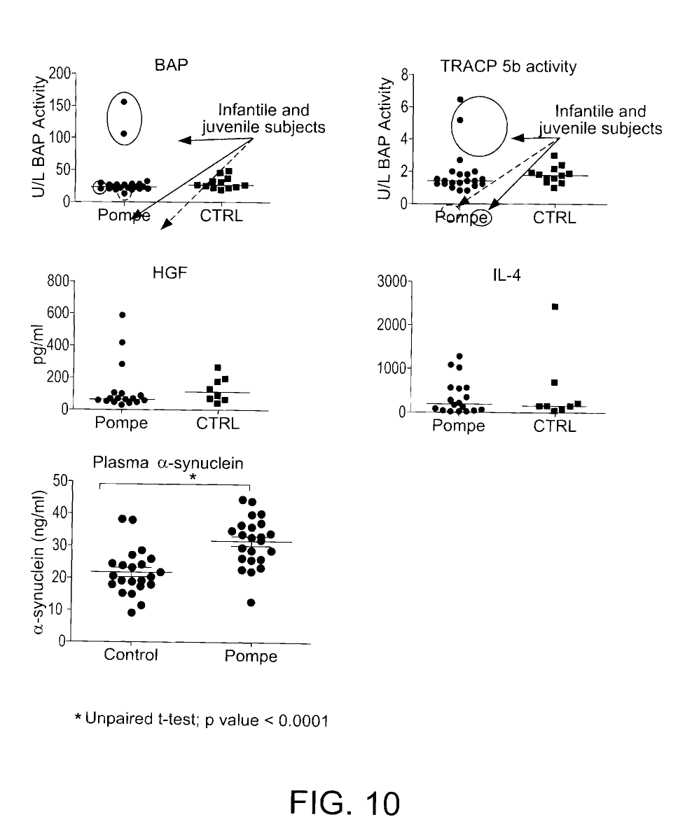

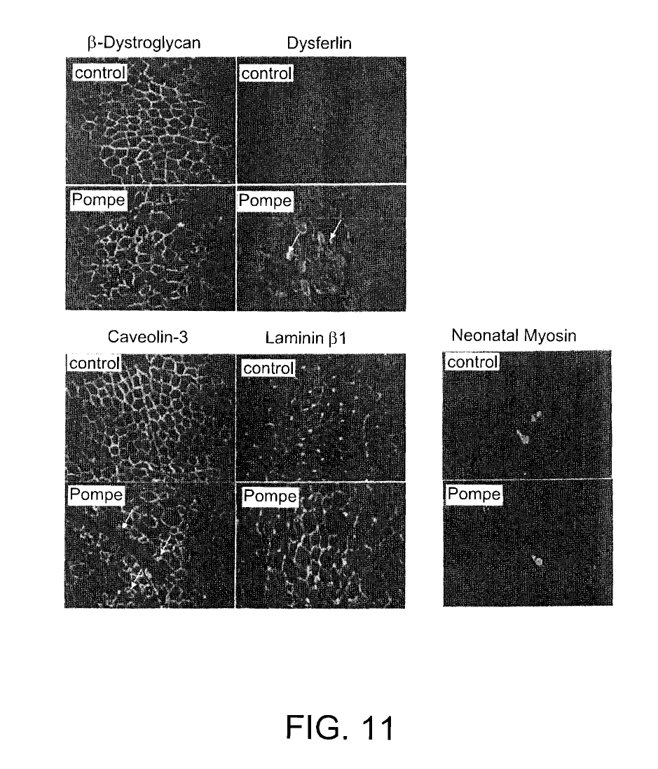

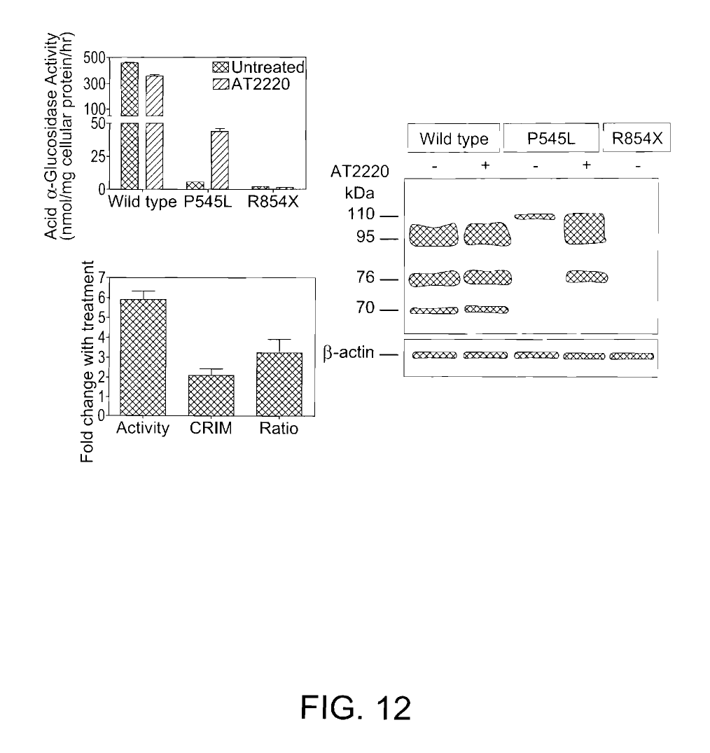

This study included Pompe patients of different genotypes. Blood was drawn immediately prior to enzyme infusion from any patients that were receiving enzyme replacement therapy. Plasma from Pompe patients was screened for potential markers associated with inflammation (cytokines), muscle regeneration, membrane integrity (collagen IV) and autophagy (cathepsin B) (FIG. 9). Plasma levels of the lysosomal marker cathepsin D and the growth factors PDGF-AA. PDGF-AA/BB and HGF were significantly (p<0.05, unpaired t-test) lower in GSD-II patients compared to controls, while cathepsin B and the cytokines MIP-1alpha and VEGF were significantly (p<0.05, unpaired t-test) higher in GSD-II patients compared to controls. Increased levels of Interleukin-6 (IL-6), increased levels of Interleukin-8 (IL-8), increased levels of Interleukin-17 (IL-17), increased levels of collagen IV, decreased levels of Interleukin-7 (IL-7), and decreased levels of Interleukin-12 p40 subunit (IL-12p40) in the Pompe patiens were also detected when compared to control individuals. Millipores Human Cytokine Lincoplex panel was used to screen plasma samples for cytokines levels. All other markers Were measured using commercially available ELISAs. Lines on graphs represent medians for the Pompe and control groups.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for monitoring a therapeutic response of a Pompe disease patient following administration of an effective amount of a specific pharmacological chaperone of acid α-glucosidase, which method comprises determining whether there is an improvement in a surrogate marker that is associated with Pompe disease.

2. A method for monitoring treatment of a patient with Pompe disease following administration of a specific pharmacological chaperone of acid α-glucosidase, which method comprises determining whether there is an improvement in a surrogate marker that is associated with Pompe disease, wherein an improvement indicates that the patient is a responder.

3. The method of claim 1, wherein the surrogate marker is a systemic surrogate marker.

4. The method of claim 3, wherein the marker is at least one selected from the group consisting of decreased lysosomal acid α-glucosidase activity; the presence of lipid-laden macrophages (“Pompe macrophages”); increased levels of cathepsin B, increased levels of Macrophage inflammatory protein 1 alpha (MIP-1 alpha), increased levels of vascular endothelial growth factor (VEGF), increased levels of Interleukin-6 (IL-6), increased levels of Interleukin-8 (IL-8), increased levels of Interleukin-17 (IL-17), increased levels of collagen IV, decreased levels of cathepsin D, decreased levels of platelet-derived growth factor AA (PDGF-AA), decreased levels of platelet-derived growth factor AA/BB (PDGF-AA/BB), decreased levels of Interleukin-7 (IL-7), and decreased levels of Interleukin-12 p40 subunit (IL-12p40).

5. The method of claim 1, wherein the surrogate marker is a sub-cellular surrogate marker.

6. The method of claim 5, wherein the sub-cellular surrogate marker is at least one selected from the group consisting of aberrant trafficking of α-glucosidase in cells from Pompe patients from the ER to the lysosome; aberrant trafficking of cellular lipids though the endosomal pathway; the presence of increased amounts misfolded α-glucosidase in the ER or cytosol; the presence of ER and/or stress resulting from toxic accumulation of α-glucosidase (as determined by gene and/or protein expression of stress-related markers); aberrant endosomal pH levels; the presence of increased plasma membrane expression of MHCII and/or CD1d on monocytes; aberrant cell morphology; suppression of the ubiquitin/proteasome pathway; and an increase in the amount of ubiquitinated proteins.

7. The method of claim 1, wherein the specific pharmacological chaperone is an inhibitor of acid α-glucosidase.

8. The method of claim 7, wherein the inhibitor is a reversible competitive inhibitor.

9. The method of claim 8, wherein the inhibitor is 1-deoxynojirimycin.

10. A method for monitoring treatment of a Pompe disease patient following administration to the patient of an effective amount of a specific pharmacological chaperone that binds to acid α-glucosidase, which method comprises determining the effect on cytoplasmic staining of a cell from the patient, wherein detection of a staining pattern in the cell that is similar to the staining pattern in a cell from a healthy individual indicates that the individual with Pompe disease is a responder.

11. The method of claim 10, wherein the cytoplasmic staining is lysosomal staining.

12. The method of claim 11, wherein the lysosomal staining is detection of the presence of α-glucosidase.

13. The method of claim 11, wherein the lysosomal staining is detection of LAMP-1 expression.

14. The method of claim 10, wherein the cytoplasmic staining is detection of polyubiquitinated proteins.

15. The method of claim 10, wherein the specific pharmacological chaperone is an inhibitor of α-glucosidase.

16. The method of claim 15, wherein the inhibitor is a reversible competitive inhibitor.

17. The method of claim 16, wherein the inhibitor is 1-deoxynojirimycin.

18. The method of claim 2, wherein the surrogate marker is a systemic surrogate marker.

19. The method of claim 2, wherein the surrogate marker is a sub-cellular surrogate marker.

20. The method of 2, wherein the specific pharmacological chaperone is an inhibitor of acid α-glucosidase.
